(19) United States
(12) Patent Application Publication
(10) Pub. No.: US 2022/0362146 A1
(43) Pub. Date: Nov. 17, 2022

(54) ADMINISTRATION OF CANNABIDIOL

(71) Applicant: GN Tobacco Sweden AB, Järfälla (SE)

(72) Inventor: Davit Hovsepyan, Yerevan (AM)

(21) Appl. No.: 17/623,369

(22) PCT Filed: Jun. 25, 2020
(86) PCT No.: PCT/EP2020/067888

§ 371 Date: Dec. 28, 2021

(30) Foreign Application Priority Data
Jun. 28, 2019 (EP) 19183337.5

(51) Int. Cl. A61K 9/00; A61K 31/05; A61K 47/38

(52) U.S. Cl. CPC A61K 9/0056; A61K 31/05; A61K 47/38

(57) ABSTRACT

There is provided a dose unit for cannabidiol (CBD) comprising a pouch of an and water insoluble and saliva penetrable packaging material comprising a moist formulation, the moist formulation comprising a carrier material, water and CBD.

### FIELD OF THE INVENTION

The following invention relates to a novel dose unit comprising cannabidiol (CBD).

### BACKGROUND

Cannabidiol (CBD) is the active component of the pharmaceutical Epidolex, which is currently approved for the treatment of seizures. Cannabidiol is also proposed to have effect as an antipsychotic and an antidepressant.

Epidolex is a solution for oral administration (to be swallowed). Administration is somewhat cumbersome since the correct volume of solution must be handled and measured. Problems include errors when measuring the volume to be administered, and spilling.

Moreover, CBD is currently frequently administered orally as oil drops, in particular in non-pharma applications. This has similar disadvantages, including problems when measuring the correct volume, and risk of spilling. Users are also frequently uncomfortable to handle the oil in public. Moreover, the oil often has a strong taste, that stays in the mouth for a long time.

Hence there is a need for improved administration of CBD, such that CBD can be administered in a manner that is convenient and effective. There is also a need for suitable manners of production, such that production is safe and cost efficient.

### SUMMARY OF INVENTION

In a first aspect of the invention there is provided a dose unit for cannabidiol (CBD) comprising a pouch of a water insoluble and saliva penetrable packaging material comprising a formulation, the formulation comprising a water insoluble carrier material, water and CBD.

The dose unit provides convenient and safe dosing of CBD. The pouches are easy to carry along, for example in a handbag, because there is no risk of leakage of liquid. People that already use Scandinavian type snuff (“snus”) will find it very easy to use.

The inventor has also found that including moistness, in particular as water, in the dose unit solves several problems. The moistness has the advantage of not drying the saliva in the mouth. This provides a more comfortable administration to the user, by softening the dosage unit, and prevents dryness in the mouth. The moistness also provides efficient transfer of CBD to the oral mucosa of the user.

The moisture of the formulation also provides dust free production of the dose unit. The moist formulation can be handled, in particular during packaging into the pouches, without special equipment for deducting or strong ventilation and other working safety measurements. Also, the product is dry enough not to stick to the machinery. Moreover, a moist formulation prevents phasing of particles of different sizes or densities while preparing the formulation, which is otherwise a problem with a dry formulation. Hence adding water makes it easier to obtain a homogenous mixture.

Water is preferred for providing Moisture. Hence, in some embodiments, water is the only component that contributes to moisture.

The moisture content of the dose unit may be from 41% to 48%, more preferably from 43% to 46%. The dose unit may comprise a humectant. This refers to the final product.

The dose unit may comprise from 40% to 47% of the carrier material and from 40% to 48% of water, more preferably from 42% to 45% of the carrier material and from 43% to 46% of water. This refers to the final product. Having this water content and moisture provides a long shelf life for the dose units, since the dose unit tends to lose water over time.

The carrier material may be a cellulose material, in particular microcrystalline cellulose. The dose unit may comprise a flavouring agent. The advantage of a flavouring agent is that the user does not taste the CBD.

The dose unit may comprise maltodextrine. Maltodextrine enhances the uptake of CBD.

In a preferred embodiment the formulation comprises CBD, maltodextrine, xanthan gum, microcrystalline cellulose, flavouring, ethanol, glycerine, propylene glycol, benzyl alcohol and water.

In an even more preferred embodiment the dose unit (i.e. final product) comprises or consists of from 1.5% to 2% CBD, from 2% to 2.4% maltodextrine, from 0.08% to 0.12% xantan gum, from 40% to 47%, more preferably from 42% to 45% of microcrystalline cellulose, from 2% to 3% flavouring, from 1% to 2% ethanol, from 1 to 2% glycerine, from 1 to 2% propylene glycol, from 0.2% to 0.5% benzyl alcohol, and water, contained in a pouch This refers to the final product

The weight of the dose unit may preferably from 0.3 g to 1 g.

In a second aspect of the invention there is provided a formulation for producing a dose unit according to the first aspect of the invention, said formulation comprising CBD, from 50% to 55% of a microcrystalline cellulose carrier material, and from 33% to 36% of water and a binding agent.

In a third aspect of the invention there is provided a method of producing a dose unit according to the first aspect of the invention comprising the steps of the steps of
a) mixing the components of the formulation,
b) filling a pouch of a water insoluble and saliva penetrable packaging material with the moist formulation and
c) spraying the filled pouch with water, to allow the filled pouch to absorb the water and to obtain a final moisture content of from 41% to 48%, more preferably 43% to 46%.

By adding water in two steps, i.e. first in the formulation and then in a final moisture adjustment step, the moisture of the formulation has suitable characteristics for handling in machinery, while providing sufficient moisture for the final product.

In a fourth aspect there is provided a dose unit obtainable according to the method of the third aspect of the invention.

In a fifth aspect there is provided the use of a dose unit according to the first or fourth aspect for use as a medicament, in particular for the treatment of pain, seizures, psychosis, psychosis or depression.

The accompanying drawings form a part of the specification and schematically illustrate preferred embodiments of the invention, and serve to illustrate the principles of the invention.

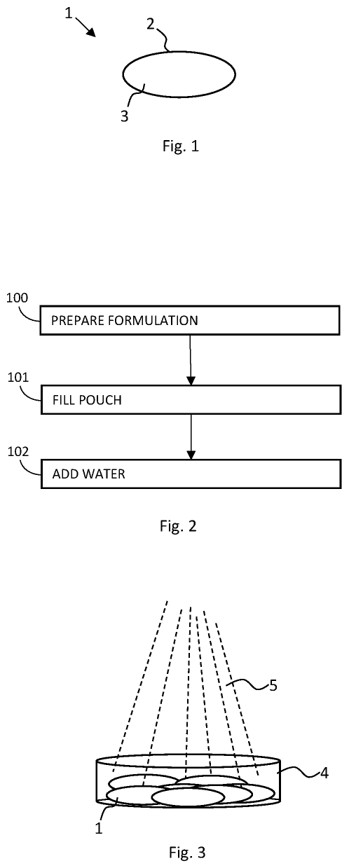

FIG. 1 is a schematic drawing of a dose unit.

FIG. 2 is a diagram that shows a method.

FIG. 3 is a schematic drawing of a container with pouches being sprayed with water.

### DETAILED DESCRIPTION

“Oral” and “oral use” means use in the oral cavity of a human, such as buccal placement.

“moisture content” refers to the total amount of oven volatile ingredients, such as for example water and ethanol, in the preparation. Moisture content measurement is carried out using a moisture analysis device by heating the sample to 120° C. and weighting the non-volatile fraction that remains in the sample when the weight has stabilized. For example, glycerine and propylene glycol does not evaporate at 120° C. and remains in the non-volatile fraction.

Percentages disclosed herein are weight/weight. Percentages of the various components in the formulation refers to the percentage in the formulation before adding any extra water at the final stage of production of the dose unit, except where indicated. Hence, “Final concentration” refers to the concentration in the final product.

With reference to FIG. 1, the dose unit 1 is intended for oral use and sized to fit discreetly and comfortable in a user's mouth between the upper or lower gum and the lip. A roughly rectangular shaped pouch is preferred where the dimensions may be from 25 to 40 mm times 10 to 20 mm. A preferred shape of the dose unit is a “pillow”-shape. The weight of the formulation in the dose unit may be from 0.3 g to 1 g, more preferably from 0.4 to 0.8 g. The weight of the packaging material is not included in this as the weight is almost negligible at 0.05-0.12 g per dose unit.

The dose unit 1 comprises a pouch 2 of packaging material that contains the moist formulation 3. The formulation 3 may be characterized as a moist granular material.

The packaging material is essentially insoluble in saliva. The packaging material is saliva penetrable. This allows the saliva to penetrate the packaging material so that CBD is released in the oral cavity. The packaging material is preferably sheet-like and wrapped to form the pouches 2. Suitable materials for the packaging material include non-woven, for example viscose. Another suitable non-woven material is cellulose fleece. The packaging material may comprise a cellulose fiber and a second polymer such as for example polyethylene. The second polymer is preferably compatible with heat sealing. The packaging material may have a micro perforation to increase release. The packaging material is preferably orally acceptable. Preferably the packaging material has a comfortable feel in the mouth. Suitable pouches 2 are furthermore described in EP3087852 and a suitable machine for producing the dosage units is described in U.S. Pat. No. 6,135,120.

All compounds and ingredients including the packaging material are preferably pharmaceutically acceptable and orally compatible. They should preferably be of pharmaceutical grade purity and free of contamination. However, the dose unit can be for pharmaceutical use or non-pharmaceutical use. The components of the formulation are preferably homogeneously mixed.

Formulation

CBD in the formulation can be provided as CBD isolate, for example CBD isolate with at least 98% purity, more preferably 99% purity, even more preferably 99.5% purity and most preferably 99.9% purity, which may be provided as a dry powder. CBD may also be provided as CBD oil or hemp oil, which preferably is free or essentially free of THC. The amount of CBD in each pouch depends on the intended use. A useful proportion of CBD may be from 0.1% to 10%, where from 1% to 3% is preferred and from 1.8% to 2.4% is most preferred. A suitable CBD dose may be from 6 mg to 100 mg in each pouch, more preferably from 10 mg to 50 mg, even more preferably from 12 mg to 20 mg.

The formulation comprises a carrier material. The carrier material is insoluble in saliva or water. The carrier is preferably able to adsorb various components of the formulation, in particular CBD. The carrier material is preferably hydrophobic. The carrier material may be in powder form. The average particle size may be from about 50 um to about 500 um.

The carrier material may be a cellulose carrier material for example selected from the group consisting of microcrystalline cellulose, powdered cellulose, or water insoluble plant fibers, or any combination thereof. Suitable plant fibers include bamboo fibers, oat fibers, maize fibers, cocoa fibers, tomato fibers, apple fibers and similar. In particular, microcrystalline cellulose is preferred. A preferred form of microcrystalline cellulose is LP200. The formulation preferably comprises 45% to 60%, more preferably from 50% to 55% of the carrier material and most preferably from 52% to 53% of the carrier material, in particular when the carrier material is microcrystalline cellulose.

The carrier material and the packaging material is selected such that the carrier material remains in the pouch even after long use (minutes to hours). Hence, preferably the packaging material is chosen so that the carrier material is retained in the pouch formed by the packaging material. For example, the pore size of the packing material is selected appropriately.

The formulation may preferably comprise a flavouring agent. Any suitable type of flavouring agent can be used such a lemon, mint, etc. The flavouring agent preferably hides the taste of CBD to at least some extent. The concentration of flavouring may be from 0.5% to 10% more preferred from 3% to 4%. The flavouring agent may be provided with a suitable solvent, for example ethanol. The amount of ethanol may be from 0.5% to 3% more preferably from 1% to 2% or slightly more to obtain the final concentration of Table 1. The amount of solvent depends on the flavouring agent that is used.

The formulation may additionally comprise a binding agent, for example xanthan gum. Other suitable binding agents include alginin, guar gum and other vegetable gums. A suitable concentration of xantan gum may be from 0.05% to 0.5%, more preferably 0.05 to 0.15% to obtain the final concentration of Table 1.

The formulation may comprise a humectant, for example glycerine. A suitable concentration may be from 1% to 2%, or slightly more to obtain the final concentration of Table 1. Alternative humectants include sucrose, triacetin and other non-ionic polyols.

The formulation may contain a preservative. Useful preservatives include alcohols such as propylene glycol and benzyl alcohol, or combinations thereof. A suitable concentration of propylene glycol may be from 1% to 2% or slightly more to obtain the final concentration of Table 1 and a suitable concentration of benzyl alcohol may be from 0.15% to 0.5% or slightly more to obtain the final concentration of Table 1.

The formulation may comprise maltodextrine. Maltodextrine enhances the uptake of CBD. A suitable concentration of maltodextrine is from 1% to 4% where from 2% to 3% is preferred.

The water content of the formulation is preferably from 30% to 40%, more preferably from 33% to 36%.

The formulation is preferably a moist formulation. The moisture content of the formulation when packed in the pouches is preferably at least 10%, more preferably at least 20%, even more preferably at least 30%. The moisture content is preferably from 20% to 50%, more preferably from 30% to 45%, even more preferably from 36% to 44%, even more preferably from 36% to −43% and most preferably from 39% to 42%.

In one embodiment the formulation comprises or consists of CBD, maltodextrine, xanthan gum, microcrystalline cellulose, flavouring, ethanol, glycerine, propylene glycol, benzyl alcohol and water.

In one embodiment, the formulation comprises or consists of from 1% to 3% CBD, from 2% to 3% maltodextrine, from 0.05% to 0.15% xantan gum, from 50% to 55%, more preferably from 52% to 53% of microcrystalline cellulose, from 3% to 4% flavouring, from 1% to 2% ethanol, from 1 to 2% glycerine, from 1 to 2% propylene glycol, from 0.15% to 0.5% benzyl alcohol, and water.

Method and Water Addition, Use

In a preferred embodiment, the final moisture content of the dose unit 1 may be adjusted by adding water to the filled pouch 3, for example by spraying the filled pouch with water, causing the filled pouch 3 to absorb the water. A suitable final moisture content of the dose unit may be from 40% to 49% more preferably from 41% to 48%, in particular from 43% to 46%.

A suitable amount of water added to each pouch (dose unit) may be from 0.08 g to 0.16 g, more preferably from 0.10 g to 0.14 g. Suitable final concentrations of the contents in the dose unit, after final addition of water, are as follows:

TABLE 1

| | | Preferred range |
|---|---|---|
| | Range (wt %) | (wt %) |
| CBD | 1.5-2.0 | 1.6-1.8 |
| Maltodextrine | 2.0-2.4 | 2.1-2.3 |
| Xantan gum | 0.08-0.12 | 0.09-0.1 |
| Carrier material, preferably | 40-47 | 42-45 |
| microcrysalline cellulose | | |
| Flavoring | 2-3 | 2.5-2.8 |
| Ethanol | 1-2 | 1.2-1.4 |
| Glycerol | 1-2 | 1.2-1.4 |
| Propylene glucol | 1-2 | 1.2-1.4 |
| Benzyl alcohol | 0.2-0.5 | 0.3-0.4 |
| Water | 40-48 | 43-46 |

Each of the concentration ranges in Table 1 are separate embodiments freely combinable with other embodiments herein.

In one embodiment the final product comprises 40-47% carrier material, 40-48% water and CBD.

Hence, in preferred embodiment the dose unit (i.e. final product) comprises or consists of from 1.5% to 2% CBD, from 2% to 2.4% maltodextrine, from 0.08% to 0.12% xantan gum, from 40% to 47%, more preferably from 42% to 45% of microcrystalline cellulose, from 2% to 3% flavouring, from 1% to 2% ethanol, from 1 to 2% glycerine, from 1 to 2% propylene glycol, from 0.2% to 0.5% benzyl alcohol, and water, contained in a pouch.

In some embodiments, it may be suitable to add all the water and other moisture contributing components in the initial mixing step to arrive at the final concentrations of Table 1. Hence, then water is not added in a second water adding step as described herein.

A dose unit 1 can be packed in a moisture preserving container 4 to prevent drying of the moist formulation. Preferably, the moisture of the dose unit 1 is preserved for several months.

The dose unit 1 is used as follows. The user places a dose unit 1 in the mouth, preferably between the upper lip and the gum, preferably offset to the right or the left in the users' mouth, in a way similar that how Scandinavian snuff, or “snus”, is used. The user allows the dose unit 1 to remain there for time period which may be from 5 minutes to 120 minutes, to allow release of CBD. After use, the pouch, which still contains the carrier material, is discarded. CBD uptake occurs through the oral mucosa and in the intestines after swallowing CBD-containing saliva.

There is also provided a method for producing a dose unit 1 as described above comprising the steps of 100 preparing formulation by mixing the components of the formulation, i.e. mixing water, CBD and the water-insoluble carrier material and possibly other components. The moisture content of the formulation, may, as described above, be at least 10%, more preferably at least 20% and most preferably at least 30%, and preferably from 36% to 43%. In step 101 pouches 2 are filled with the formulation 3 and then sealed. The pouch 2 may be sealed with any suitable method for example heat sealing or using a suitable glue. Water is then, in step 102, added to the filled pouch, for example by spraying the filled pouch with water, to allow the dose unit 1 to absorb the water and to obtain a final moisture content that preferably is higher than the initial moisture content, preferably of from 40% to 49% more preferably from 41% to 48%, in particular from 43% to 46%. Water may be added with other methods such as injecting the pouch or dipping the pouch. In one embodiment a plurality of dose units 1 filled with the formulation 3 placed in an open container 4 and a predetermined amount of water is added to the pouches in the container, for example by spraying 5 the pouches with water in the container as shown in FIG. 3. When the water has been added, the container is closed and sealed to some extent, to prevent the water from evaporating. However, since complete water tight containers are expensive a seal and a container that is not completely water tight may be used. The water added will then equilibrate to distribute evenly between the pouches. For example, a lid may be placed on the container 4 and then sealed to container 4.

The correct amount of water to be added can be determined using experimentation and adjustment of the method of adding water, for example by adjusting the amount of water sprayed on the pouch 2. A moisture analyser is suitably used for this step.

The inventor has found that adding all moisture to the formulation initially, a formulation that was sticking to the machinery somewhat was obtained. By not adding some the water to the formulation after filling the pouches, this problem was avoided.

Medical Use

The dose unit 1 can be used for administering CBD to a person. Hence there is provided a method of treatment comprising the step of administering a dose unit 1 to a person, and a dose unit 1 for use in medicine. The dose of CBD administered to the patient and the administration scheme of CBD can be selected by a skilled person.

The person may be patient that is suffering from an illness. The dose unit is useful for treatment of for example pain, seizures, psychosis, psychosis or depression.

### Example 1

A moist formulation was prepared as follows.

| | | |
|---|---|---|
| CBD Isolate 99% | 2.104 | g |
| Maltodextrine | 2.629 | g |
| Xanthan gum | 0.105 | g |
| Microcrystalline cellulose LP200 | 52.576 | g |
| Flavouring | 3.312 | g |
| Ethanol | 1.577 | g |
| Glycerin | 1.577 | g |
| Propylene glycol | 1.577 | g |
| Benzyl alcohol | 0.368 | g |
| Purified water | 34.175 | g |

| | |
|---|---|
| Total | 100.000 |

All dry ingredients were well mixed. All liquid ingredients except water were well mixed with the dry ingredients. The water was then applied with pressurized water jets to provide homogenous mixing.

The preparation was found to have suitable moisture to avoid dust formation. The formulation was easily handled in machines and did not stick to the machinery.

### Example 2

The formulation from Example 1 was packed in pouches made of cellulose fleece and polyethylene using an in line sealer in which the paper formed a tube, which was filled with the formulation. Each pouch had a size of 34 mm×14 mm and contained approximately 0.63 g of the formulation, containing approximately 13 mg of CBD. The pouches were sealed using an in line hot sealer, to obtain dose units. The formulation was easily handled in machines and did not stick to the machinery.

### Example 3

The pouches were weighted and sprayed with water to adjust the final weight of the dose unit. Approximately 0.12 g of water was added to each pouch. The moisture content of the pouches was analysed in a moisture analyser at 120° C. The moisture content was between 43% and 46% in different measurements.

### Example 4

The dose units were tested by a test person as follows. One dose unit was placed under the upper lip between the lip and the gum to the side for 20 minutes. The test person reported that she felt the effect of CBD within 5-10 minutes and that she did not feel dryness in the mouth. The test person did not taste the CBD.

### Example 5

Several dose units were stored in a container that was closed but not completely waterproof or vapor proof for several months. After several months of storage, the dose units were still moist and had retained their softness.

While the invention has been described with reference to specific exemplary embodiments, the description is in general only intended to illustrate the inventive concept and should not be taken as limiting the scope of the invention. The invention is generally defined by the claims.

1. A dose unit for cannabidiol (CBD) comprising a pouch of a water insoluble and saliva penetrable packaging material comprising a formulation, the formulation comprising a water insoluble carrier material, water and CBD.

2. The dose unit according to claim 1 comprising from 40 wt % to 47 wt % of the carrier material and from 40 wt % to 48 wt % of water.

3. The dose unit according to claim 1 comprising from 42 wt % to 45 wt % of the carrier material and from 43 wt % to 46 wt % of water.

4. The dose unit according to claim 1 where the water is from 41 wt % to 48 wt %.

5. The dose unit according to claim 1 where the water is from 43 wt % to 46 wt %.

6. The dose unit according to claim 1 comprising a humectant.

7. The dose unit according to claim 1 where the carrier material is a cellulose material.

8. The dose unit according to claim 7 where the cellulose material is microcrystalline cellulose.

9. The dose unit according to claim 1 where the formulation comprises a flavouring agent.

10. The dose unit according to claim 1 where the formulation comprises maltodextrine.

11. The dose unit according to claim 1 where the formulation comprises CBD, maltodextrine, xanthan gum, microcrystalline cellulose, flavouring, ethanol, glycerine, propylene glycol, benzyl alcohol and water.

12. The dose unit of claim 11 comprising from 1.5 wt % to 2 wt % CBD, from 2 wt % to 2.4 wt % maltodextrine, from 0.08 wt % to 0.12 wt % xantan xanthan gum, from 40 wt % to 47 wt %, more preferably from 42 wt % to 45 wt % of microcrystalline cellulose, from 2 wt % to 3 wt % flavouring, from 1 wt % to 2 wt % ethanol, from 1 wt to 2 wt % glycerine, from 1 wt % to 2 wt % propylene glycol, from 0.2 wt % to 0.5 wt % benzyl alcohol, and water.

13. The dose unit according to claim 1 having a weight of from 0.3 g to 1 g.

14. A formulation for producing a dose unit according to claim 1 comprising: CBD, from 50 wt % to 55 wt % of a microcrystalline cellulose carrier material, from 33 wt % to 36 wt % of water, and a binding agent.

15. A method for producing a dose unit according to claim 1 comprising the steps of:
a) mixing components comprising: a water insoluble carrier material, water and CBD, forming a moist formulation,
b) filling a pouch of a water insoluble and saliva penetrable packaging material with the moist formulation and
c) spraying the filled pouch with water, to allow the filled pouch to absorb the water and to obtain a final moisture content of from 43 wt % to 46 wt %.
